(19) United States
(12) Patent Application Publication
(10) Pub. No.: US 2021/0213220 A1
(43) Pub. Date: Jul. 15, 2021

(54) CAPTURE OF XENON FROM ANAESTHETIC GAS AND RE-ADMINISTRATION THEREOF TO THE PATIENT

(71) Applicant: SAGETECH MEDICAL EQUIPMENT LIMITED, Surrey (GB)

(72) Inventor: Sebastian Matthew BROWN, Totnes Devon (GB)

(21) Appl. No.: 17/059,850

(22) PCT Filed: May 28, 2018
(86) PCT No.: PCT/GB2019/051451

§ 371 Date: Nov. 30, 2020

(30) Foreign Application Priority Data
May 29, 2018 (GB) 1808745.2

(51) Int. Cl. A61M 16/00; A61M 16/08; A61M 16/10; A61M 16/20; B01D 15/40

(52) U.S. Cl. CPC A61M 16/0093; A61M 16/0891; A61M 16/104; A61M 16/209; B01D 15/40; A61M 2202/0291; B01D 2259/4533; A61M 2202/0225; B01D 2253/108; B01D 2253/1122; B01D 2253/204; B01D 2253/25; B01D 2257/11; A61M 2202/0028

(57) ABSTRACT

A method for the extraction of xenon gas bound to a filter material using supercritical CO₂ to form a mixture in which both CO₂ and xenon are in a supercritical state.

### TECHNICAL FIELD

The present invention relates to methods and systems for capturing and recycling xenon. In particular, the present invention relates to methods and systems for capturing and recycling xenon when used as an anaesthetic or neuroprotective agent in medical environments.

### BACKGROUND

Xenon is a noble gas element with uses in lasers, lighting and in medicine. In anaesthesia, xenon at concentrations of 72% in oxygen can deliver a depth of anaesthesia consistent with surgery. Xenon has been suggested to offer neuroprotective effects via inhibition of NMDA receptors and is used for neonates with birth-induced brain injury and potentially for patients following subarachnoid haemorrhage.

Xenon is a rare element, occurring at approximately 1 part per 11.5 million in air. The majority is produced as a by-product of the fractional distillation of air to form oxygen and nitrogen. However worldwide production is still very small when compared to the potential needs of anaesthesia. Therefore, significant interest exists for technology that is capable of reprocessing xenon in medical devices for anaesthesia.

Prior art focuses on the removal of xenon from oxygen during the cryogenic processing of air using selective absorbents and/or catalysis to remove hydrocarbon contaminants. Absorbents can be silica gel, zeolites, metal doped (e.g. Silver/Lithium) or more recently metal-organic frameworks. Once absorbed, the xenon can be removed by freezing out at cryogenic temperatures or by heating and evacuation with a gas (helium or nitrogen). These processes form part of the cryogenic separation process. Cryogenic processes require very significant capital infrastructure, only being economically viable in large scale and to produce multiple products (e.g. the separation of air). However, this capital-intensive technology is not suitable for the remanufacture of xenon from medical use.

Gas-chromatography has been proposed as a non-cryogenic purification methodology (CN102491293B) to separate xenon from krypton. By this method, helium or nitrogen driving gas is used to pass xenon gas through a gas chromatography column. Due to the strong interaction of the xenon with the column stationary phase, the passage of the Xenon is retarded compared to krypton and other contaminants. The xenon can then be extracted from the driving gas following elution from the top of the column. Gas purification systems suffer from low production rates due to low density and the batch nature of chromatographic processes. Furthermore, purified products must be separated from the driving gas, which is often just as complicated as the original separation.

Limited recirculation measures are used in anaesthesia to preserve volatile anaesthetics. However, even these rebreathing systems (e.g. circle system) run at low-flow conditions of 0.5 L/min fresh gas flow will only lead to 20% of the administered xenon being absorbed by the patient. Therefore, the entire world production of xenon would only be sufficient for 400,000 anaesthetics. 4 million anaesthetics are delivered each year in the UK alone. Therefore, technologies outside or in addition to rebreathing systems are required. Ideally these systems would be incorporated into the anaesthesia device as it is likely that even with very high-efficiency systems, xenon use would be restricted to certain patients/long cases. Currently, xenon is operated in an intensive care setting for long-term use. Therefore, it is likely that local recycling systems, either at the patient or within the hospital itself would be most economical.

### DESCRIPTION

According to an aspect of the present invention there is provided a method for the extraction of xenon gas bound to a filter material using supercritical carbon dioxide to form a mixture in which both carbon dioxide and xenon are in a supercritical state.

The present invention also provides a method of recovering xenon anaesthetic agent from a filter, comprising the step of subjecting the filter to a supercritical fluid, thereby forming a supercritical solution.

The present invention also provides a method for extraction of xenon by supercritical carbon dioxide by first capturing xenon from the exhaust of a medical device delivering xenon by binding it to a filter material that may include but is not limited to silica gel, zeolites, metal organic frameworks, or metal doped silica/zeolite.

The present invention also provides a method for capturing xenon from the exhaust of a medical device delivering xenon by binding it to a filter material formed of a silver or lithium doped aerogel.

The present invention also provides a method to capture xenon from exhausted anaesthetic gas, the method comprising processing gas containing xenon with filter material.

The method may further comprise the step of releasing xenon from the filter using a supercritical fluid.

Methods formed in accordance with the present invention may further comprise the steps of: passing gas derived from a patient in a medical environment through a filter so that xenon anaesthetic agent becomes bound thereto; subjecting the filter material to a supercritical fluid, thereby forming a supercritical solution; removing contaminants from the supercritical solution; collecting the xenon anaesthetic agent from the supercritical solution; and reintroducing the xenon anaesthetic agent to a patient.

The present invention also provides apparatus to or suitable to perform a method as described herein, comprising a module housing filter material and into which anaesthetic gas can pass so that xenon anaesthetic agent binds to the filter material, and a supercritical fluid source, the module being resistant to supercritical fluid and able to withstand supercritical pressure and temperature so as to enable captured xenon to be reclaimed by exposure to supercritical fluid.

The present invention also provides for the separation of xenon gas derived from a medical device and carbon dioxide using a vortex tube.

The present invention also provides a method of producing medical grade xenon from contaminated xenon derived from the exhaust of a xenon delivery medical device by using liquid carbon dioxide chromatography followed by separation of xenon from carbon dioxide.

The present invention also provides a method in which liquid CO₂ is used as the mobile phase for chromatographic purification of xenon from gaseous contaminants derived from the patient or breathing systems.

The purpose of some aspects and embodiments of this invention is to provide a method for high volume, high purity xenon recycling for medical devices by using carbon dioxide in liquid and supercritical phases for the extraction and purification and re-delivery of xenon to medical devices used in anaesthesia.

Apparatus formed in accordance with the present invention may comprise a chamber containing an absorbent which may include but is not limited to silica gel, zeolites, metal organic frameworks, or metal doped silica/zeolite, most preferably a metal (silver or lithium) doped aerogel is attached to the exhaust of the anaesthetic machine. The anaesthetic exhaust contains xenon at 1-100%, most preferably containing clinically relevant concentrations such as 45% for hypnosis and 72% for anaesthesia usually in oxygen. This is contaminated by hydrocarbons and many other compounds present in exhaled breath (e.g. ethanol, acetone) and from the machine/gases (e.g. hydrocarbons, plasticisers). This absorbent selectively absorbs the xenon gas and some contaminants, but oxygen passes through. Preferably, binding of the xenon to the absorbent can be increased by pressurizing and/or cooling the exhaust gases into the capture cylinder.

The present invention provides a method for extraction of xenon by supercritical CO₂ by first capturing xenon from the exhaust of a medical device delivering xenon by binding it to a filter material that may include but is not limited to silica gel, zeolites, metal organic frameworks, or metal doped silica/zeolite.

The present invention provides a method for capturing xenon from the exhaust of a medical device delivering xenon by binding it to a filter material formed of a metal (silver or lithium) doped aerogel.

The present invention provides a method for the capture and extraction of xenon by supercritical CO₂ by first capturing xenon onto a filter material in a container, wherein the xenon reversibly binds to the filter material. The container may be connected to the exhaust port of an anaesthetic machine or medical device so that waste gas containing xenon is passed through the filter material in the container to bind the xenon gas from the waste gas stream. In a preferred embodiment of the invention, the container is then disconnected from the anaesthetic machine or medical device exhaust and connected to a source of supercritical CO₂. Supercritical CO₂ is passed through the container, causing the xenon to be released from the filter material and leave the container with the supercritical CO₂. In this preferred embodiment the container is tolerant of pressures in excess of the critical pressure of carbon dioxide (73 bar). In another embodiment of the invention, the container may not be pressure-tolerant to the critical pressure of carbon dioxide, but it is placed inside a container that is pressure-tolerant above the critical pressure of carbon dioxide during supercritical fluid extraction.

The present invention also provides a method for the capture of xenon from a medical device wherein the xenon-containing gas stream is first exposed to a filter material in a container that reversibly binds xenon, the container being subsequently disconnected from the medical device and connected to a source of supercritical carbon dioxide for extraction of the xenon gas by supercritical CO₂.

In an embodiment of the invention the container has a port at either end that allows the ingress and egress of first xenon-containing gas from the medical device and second supercritical fluid. In another embodiment of the invention, separate ports at each end can be used first for the ingress and egress of xenon-containing gas and second supercritical fluid. In a further embodiment of the invention, the ingress and egress ports may be different in shape or size so that they can only be connected to the medical device or to the source of supercritical fluid in a specified orientation. By this method, gas containing xenon would first enter the container from one end of the container until the container was disconnected from the medical device and then, second the supercritical fluid enter from the other end of the container when connected to the source of supercritical fluid. This method allows the binding of xenon to the filter material in one direction and the extraction of xenon by supercritical fluid in the opposite direction. This counter-current loading and unloading of xenon from the filter material offers an efficiency improvement to loading and unloading the filter material with xenon in the same direction.

The present invention also provides a container with a single ingress and egress port at either end first for the passage of xenon-containing gas to bind to the filter material in the container and second, for the passage of supercritical fluid through the container to remove xenon from the filter material.

The present invention also provides a container with two ports at either end, one port at either end first for the ingress and egress of xenon-containing gas to bind to the filter material in the container and another port at either end for second, the extraction of xenon from the filter material by the ingress and egress of supercritical fluid.

The present invention also provides a container with different single or double ports at either end, so that first xenon-containing gas can be passed through the container and bind to the filter material in one direction and second so that supercritical fluid can pass through the container in the opposite direction and extract xenon from the filter material.

Some aspects and embodiments relate to the remanufacture of xenon gas for medical devices.

In an embodiment of the invention, the absorbent is regenerated by passing supercritical carbon dioxide through the container and absorbent. Carbon dioxide becomes a supercritical fluid above its critical temperature (31 degrees Celsius) and pressure (73.8 bar). At this critical point, carbon dioxide has the properties of both a gas and a liquid. It expands to fill the container it is in and dissolves non-polar compounds like a liquid. This is due to the rapid increase in density at the critical point. Liquid carbon dioxide can also be used for the extraction of xenon at temperatures below the critical temperature. Supercritical fluids dissolve each other perfectly. The critical point of xenon is 17 degrees Celsius and 59 bar. Therefore, at the critical point of carbon dioxide, xenon will also be a supercritical fluid.

The present invention provides a method for the extraction of xenon gas bound to a filter material using supercritical carbon dioxide to form a mixture in which both carbon dioxide and xenon are in a supercritical state.

Another aspect of the invention provides a method for the extraction of xenon gas bound to a filter material using liquid CO₂ to form a mixture in which the xenon is in a supercritical state and the carbon dioxide is in a liquid state.

A further aspect of the invention provides a method for the extraction of xenon gas bound to a filter material using liquid CO₂ to form a mixture in which the xenon is in a liquid state and the carbon dioxide is in a liquid state.

In another aspect of the invention, the mixture of xenon and carbon dioxide exits the chamber through the exit port and through the back-pressure regulator and is subsequently depressurized into a vortex tube. The pressure drop is controlled by a pressure-regulating valve situated prior to the vortex tube. The depressurized gas enters the vortex tube tangentially, creating a vortex that is partially reflected by a throttle valve at the other end of the vortex tube. The increased kinetic energy of the high-density xenon (density 5.894 g/L at standard temperature and pressure) forces the xenon to the outside of the vortex tube, whereas the lower density carbon dioxide (density 1.964 g/L) is maintained in the centre the vortex tube and is reflected to leave at the injection end of the vortex tube. The carbon dioxide is cooled during the process and is then re-compressed and used again to extract more xenon in a circular process. Vortex tubes can be used in sequence with slightly different or the same dimensions to improve collection purity and yield of xenon.

The present invention also provides for the separation of xenon gas and carbon dioxide by the use of a vortex tube.

The present invention also provides for the separation of xenon gas and carbon dioxide using a plurality of vortex tubes arranged in series to increase the purity of the xenon-rich gas stream.

In an embodiment of this invention, the vortex tube is used to separate xenon from oxygen following cryogenic separation of air

In one embodiment of the invention, the xenon is passed through soda lime to absorb any remaining carbon dioxide and is then administered to the patient via the anaesthesia machine breathing system. The xenon exiting the vortex tube can be maintained at pressure to fill an injection chamber for controlled release into the breathing circuit as dictated by xenon detectors and a closed-loop controller present in the anaesthetic machine. This system could be used for the re-administration of anaesthetic to the same patient in a closed loop.

In a further embodiment of the invention, soda lime or another CO₂ absorbent selective for CO₂ over xenon is used to remove carbon dioxide from xenon without the use of a vortex tube.

The present invention also provides a method for the removal of carbon dioxide from xenon gas derived from a medical device first captured onto a filter material and subsequently extracted from the filter material by supercritical carbon dioxide.

The present invention also provides a method for the re-administration of xenon gas to the same patient by the capture of xenon onto a filter material, extraction of xenon in supercritical carbon dioxide, separation and removal of carbon dioxide from the xenon gas and redelivery of the xenon to the breathing circuit within the medical device for administering xenon to the patient.

The use of supercritical extraction conditions enables a much higher throughput than gas chromatography due to the high density of supercritical solutions. Furthermore, the extraction conditions are at near room temperature (31 degrees Celsius) and at pressures that, although are high (73 bar or more), these are pressures common in anaesthesia using bottled oxygen. Due to the presence of bottled oxygen, and high oxygen fractions delivered to patients, elevated temperatures and flammable conditions are avoided. Due to pressurized conditions, the equipment is small and can fit into the space available for conventional anaesthesia equipment.

The use of the vortex tube allows the use of a small, zero maintenance component to achieve gas separation. The pressure drop used to drive separation is already available from the depressurization of the supercritical mixture, making this an efficient process.

In a further aspect of this invention, the depressurization of carbon dioxide can be used as a source of cooling to reduce the temperature of the exhaust gases from the anaesthetic machine entering the collection chamber. This means that industrial chilling equipment may not be required for the medical device.

The present invention also provides a system in which the cooling of exhaust gases from the xenon medical device, to improve the efficiency of xenon binding to the filter material, is achieved by the adiabatic expansion of carbon dioxide following depressurization after supercritical fluid extraction.

The use of carbon dioxide to drive the process enables the use of conventional carbon dioxide absorber used in anaesthesia re-breathe systems to purify the xenon. Furthermore, medical carbon dioxide is commonly available and carbon dioxide concentration is routinely detected as part of the gas monitoring systems on anaesthesia machines. This leads to a significant factor of safety when compared to nitrogen or helium that are not routinely of medical grade or monitored as part of anaesthesia or ICU care. Finally, pure carbon dioxide is very selective, only dissolving non-polar molecules. Therefore, significant purification of the exhaust is achieved by selective binding and desorption by supercritical carbon dioxide.

It may be necessary to purify the xenon during use due to the accumulation of contaminants. Furthermore, when the patient is woken, the xenon must be captured and processed ready for use by another patient. Purification can be driven using liquid carbon dioxide chromatography. Although supercritical carbon dioxide chromatography can be used, the liquid phase has performance benefits partly because both the xenon and carbon dioxide are not in the supercritical phase at the same time.

The present invention also provides a method in which liquid carbon dioxide is used as the mobile phase for chromatographic purification of xenon from gaseous contaminants derived from the patient or breathing systems.

Captured and extracted xenon as described previously is liquified and delivered to a chromatography column with a silica stationary phase of 5-10 microns, although other normal and reverse stationary phases can be used as familiar to those skilled in the art. Liquid carbon dioxide at a pressure of 1 to 200 bar and temperatures of less than 31 degrees Celsius and above −80 degrees Celsius can be used as the mobile phase, to drive the chromatographic separation of xenon from contaminants. Most preferably, a temperature of 10 degrees Celsius and a pressure of 70 bar is used. Xenon interacts with the silica stationary phase and its flow is retarded to a different degree to the various contaminants. Upon elution from the column the xenon is detected by mass spectrometry, microthermal (katharometer), x-ray absorption, ultrasound or refractometry although other detection systems know to those familiar with the art can be used. In the case of integration into the xenon anaesthesia machine, the xenon detector used for the measurement of the concentration of xenon in the anaesthetic circuit can also be used for the detection of chromatography product.

The present invention also provides for the use of mass spectrometry, microthermal (katharometer), x-ray absorption, ultrasound or refractometry methods to detect purified xenon produced by carbon dioxide liquid chromatography.

The detector signal is used to control a three-way valve that directs the Xenon/CO₂ mixture to a vortex tube for separation of Xenon from carbon dioxide as described previously. More than one vortex tube can be used to produce high purity Xenon. Carbon dioxide from the chromatography process is scrubbed of contaminants using a silica and activated carbon absorbent, pressurized and used again. The high purity xenon has any remaining carbon dioxide absorbed by a CO₂ absorber such as soda lime to produce medical-grade Xenon.

The present invention also provides for the re-pressurization and recirculation of carbon dioxide with/without any remaining xenon during supercritical fluid extraction and liquid carbon dioxide chromatography.

Steps to prevent microbiological transmission can be implemented at many stages of the process and are familiar to those skilled in the art. It is possible to use liquid carbon dioxide chromatography to purify xenon derived from a medical device by methods other than supercritical fluid extraction and known to those skilled in the art, including but not limited to cryogenic liquefaction and inert gas extraction at non-supercritical conditions (e.g. nitrogen and helium).

In one embodiment of this invention, the xenon can be returned to the same patient by incorporating the liquid carbon dioxide chromatography system into the medical device that delivers xenon to the patient. In another embodiment of this invention, the medical grade xenon can be used for other patients following the normal pharmaceutical regulatory processes for the manufacture and sale of a medicine. This would involve the purification process being delivered in a GMP (Good Manufacturing Practice) environment remote to the medical device. In one embodiment of this invention, the capture and extraction of xenon is performed by the medical device delivering xenon to the patient and then this extracted xenon is transported to a GMP facility for purification and subsequent release as a medicine.

The present invention also provides for the production of medical grade xenon from contaminated xenon derived from the exhaust of a xenon delivery medical device by using liquid carbon dioxide chromatography followed by separation of xenon from carbon dioxide.

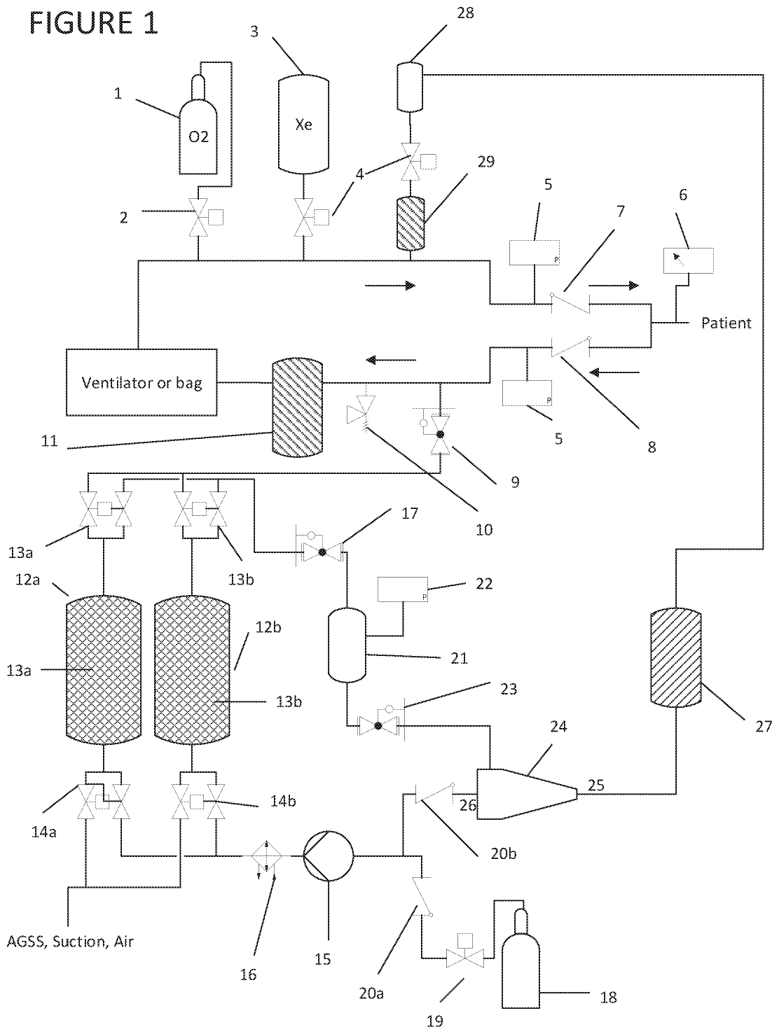

Furthermore, it is anticipated that the steps of capture and extraction may be separated. In a further embodiment of the invention, a semi-pressure intolerant sleeve is used to house the filter material as described previously. This sleeve is made of a stainless-steel tube which can tolerate extraction pressures up to 80-100 bar working pressure. The ends of the tube are made of plastic and are allowed limited movement. A seal such prevents gas leaking between the plastic caps and the stainless-steel tube. A connector links the canister to the exhaust of the anaesthetic circuit and this exhaust may be cooled and the canister may be cooled to improve binding. The pressure vessel that houses the canister for the purposes of extraction has mouldings that fit the cap at either end. During extraction, when pressurized carbon dioxide enters the canister, the caps move outwards slightly, retained by the ends of the pressure vessel and carbon dioxide can flow through the canister only due to seals in the pressure vessel that operate between the cap and pressure vessel and the seals between the canister tube and plastic ends. This system overcomes the problems inherent in manufacturing canisters for atmospheric pressure gas collection if they are also required for high pressure extraction. These canisters would be too large to use or be economical if they were the principal pressure vessel. In the system in FIG. 1, the chambers can be small because capture and extraction are happening frequently. However, when capture and extraction are separated, the canister needs to be large to hold enough xenon to make transport economical and therefore wall tensions are higher, and the end pressures can be very high—over 4 tonnes. In this system, the stainless-steel tube maintains pressure well as the hoop stress and is thin walled as it is contained within the housing, it only needs a factor of safety of 1.5. The ends are essentially free-floating and therefore the pressure is held by the ends of the pressure vessel rather than the junction between the tube and caps. By this method, the pressure inside the canister is maintained and gas can flow only through the canister. In other systems with pressure-intolerant canisters, gas is required outside the canister to balance the trans-mural pressure, which can lead to gas passing outside the canister and picking up contaminants that have transferred from the operating environment, which are not controlled.

The present invention also provides the use of a pressure tolerant stainless-steel tube with sealed, floating end caps to contain the filter material as a canister so that upon pressurization above the critical pressure of carbon dioxide, the end caps move and are retained by a pressure vessel ends and flow of supercritical carbon dioxide is maintained within the canister.

The limited abundance of xenon means that the use of xenon for general anaesthesia will be limited and drug use restricted to those patients who would benefit from its neuroprotective effects, such as neonatal hypoxic encephalopathy, hypoxic encephalopathy following cardiac arrest, cardiac surgery, sub-arachnoid haemorrhage, stroke and traumatic brain injury, although other indications requiring neuroprotection are envisaged. Such situations require long-term use of xenon and are often delivered on intensive care units that do not have access to anaesthesia gas scavenging. Therefore, xenon delivery medical devices need to be capable of capture and re-delivery of xenon to the patient, purification of the patient gas volume to remove exhaled contaminants and contaminants derived from the breathing circuit/systems, and when the xenon is stopped and ‘washed out’, the capture and processing of xenon for use by another patient.

Some aspects and embodiments of this invention can serve all three scenarios. It is able to capture and re-deliver xenon to the medical device without dedicated purification. It is able to capture and purify xenon for re-delivery to the same patient as part of the medical device. It is able to purify xenon separately from the medical device as part of a process that is fit for regulations under the medicines act so that the product can be delivered to another patient.

The invention uses pressurized systems that, when combined are thermodynamically efficient- using pressure changes that are required as part of the system to drive separation. All components are small due to pressure and require minimal cooling as this is often provided by depressurization of the working fluid.

Different aspects and embodiments may be used together or separately.

### DETAILED DESCRIPTION

The present invention is more particularly shown, by way of example, with in the accompanying drawings.

The example embodiments are described in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed. On the contrary, all modifications, equivalents, and alternatives falling within the scope of the appended claims should be included. Elements of the example embodiments are consistently denoted by the same reference numerals throughout the drawings and detailed description where appropriate.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

One of ordinary skill in the art will appreciate the many possible applications and variations of the present invention based on the following examples of possible embodiments of the present invention.

FIG. 1 shows a xenon closed circle breathing system with capture, extraction and re-delivery of xenon into the circuit.

It is anticipated that although this system described in FIG. 1 is applied to a circle system, the same system can be used to deliver recycled xenon to the gas stream in other anaesthesia systems such as a reflector system or cardiopulmonary bypass machine oxygenator.

A xenon delivery medical device can be a circle system, reflector or cardiopulmonary bypass machine oxygenator.

Oxygen 1 is delivered to the anaesthetic circuit through a servo valve under electronic control 2. Xenon gas 3 is delivered to the circuit though a solenoid or piezo injection valve 4 under electronic control. Electronic control (not shown) of a negative feedback loop with a target concentration set by medical personnel is determined by pressure 5 and gas monitoring 6 systems. The oxygen/xenon gas mixture passes down the inspiratory limb of the circuit through the inspiratory one-way valve 7. The gas monitoring system detects the concentration of xenon, carbon dioxide and Oxygen at the patient end of the circuit. This is performed by a negative pressure system removing a constant stream of gas from the patient y-piece. Most of this gas is returned to the patient circuit (not shown). Expiratory gases pass down the expiratory limb to the expiratory one-way valve 8 and pressure transducer 5. This reading is used to set the back pressure of the exhaust valve 9. A pressure relief valve 10 protects the circuit from overpressure. Some of the expiratory gases are vented through the exhaust valve (adjustable pressure limiting valve) 9 and the remainder pass through the carbon dioxide absorber 11 and to the ventilator/bag assembly where either mechanical (ventilator) or manual (bag) means are used to pressurize the circle during the ventilation cycle to produce inspiration and expiration. These recirculated gases then circulate back to the inspiratory limb via the gas injectors, where further gas can be added to regulate the system volume (and therefore pressure) and gas concentrations.

Exhaust gas from the exhaust valve 9 passes down the exhaust limb to one of two collection chambers 12a 12b tolerant of supercritical carbon dioxide pressure above 73 bar. In one preferred embodiment the working pressure of the chamber is 100 bar and the vessel is manufactured from 316 stainless steel. Each collection chamber is controlled by two selection valves 13a 13b and two section valves 14a 14b. These selection valves ensure that each chamber is either set to receive gas from the exhaust valve 9 and ventilate it to air, the suction or Anaesthetic Gas Scavenging System (AGSS) or to receive supercritical carbon dioxide from the pump 15 and heater 16 and pass it to the back-pressure regulator 17. The chambers 12a 12b can have a single input and output through which both exhaust and supercritical fluid can pass or can have separate inputs for the exhaust and supercritical fluid. In a preferred embodiment, separate inputs and outputs are used for the supercritical fluid and exhaust due to the different pressures and flow-rates required for exhaust and supercritical fluid. The selection valves 13a 13b 14a 14b ensure that each chamber 12a 12b is only open to either the exhaust or the supercritical fluid and that one chamber 12a or 12b is exposed to the exhaust while the other chamber 12b or 12a is exposed to the supercritical fluid. The control of the valves is under electronic control (not shown). The flow of exhaust gas and supercritical fluid can be in the same direction or in a preferred embodiment, in different directions as shown in FIG. 1. This improves the rate of desorption of the xenon by the supercritical fluid and increases the absorption capacity.

The use of a chamber for the capture of xenon onto a filter material that is capable of withstanding pressures above the critical pressure of carbon dioxide.

The use of two chambers, such that one is exposed to the exhaust of the xenon delivery medical device and the other is exposed supercritical carbon dioxide for extraction.

The use of a single opening at either end of the chamber for the passage of both the exhaust from the xenon delivery medical device and supercritical carbon dioxide for extraction of xenon from the filter material contained within the chamber.

The use of separate openings at either end of the chamber, one for the passage of the exhaust from the xenon delivery medical device and the other for the passage of supercritical carbon dioxide for extraction of xenon from the filter material contained within the chamber.

The chambers 12a 12b are filled with a filter material 17a 17b that absorbs xenon gas. The chambers may be cooled, and the exhaust gas cooled to temperatures from room temperature down to −50 degrees Celsius (not shown) to improve binding. The filter material may include but is not limited to silica gel, zeolites, metal organic frameworks, or metal doped silica/zeolite, most preferably a metal (silver or lithium) doped aerogel. The filter material binds the xenon gas reversibly from the exhaust gases from the exhaust valve 9 when the chamber is connected to the exhaust and releases the xenon gas when exposed to the flow of supercritical carbon dioxide.

Carbon dioxide is provided by a pressurized cylinder 18 and powered valve 19 and one-way valve 20a to a pump 15 that pressurizes the carbon dioxide above 73 bar, although lower pressures can be used for liquid carbon dioxide extraction. The liquid is then heated above the critical temperature by a heater 16 to form a supercritical fluid. The supercritical fluid is exposed to the filter material 13a or 13b in pressure-tolerant chamber 12a or 12b, dissolving the xenon to form a supercritical solution. Any non-polar contaminants from the patient or breathing systems may also be absorbed by the filter material and desorbed by the supercritical solution. The supercritical solution passes to the back-pressure regulator 17 and is depressurized into a volume buffer vessel 21 with pressure monitoring 22. The supercritical solution is depressurized further through a pressure reducing valve 23 to enter the vortex tube gas separator through an inlet throttle restriction in the vortex tube 24. The tangential entry and depressurization at the throttle restriction combined with the gas reflection at the throttle valve at the xenon outlet end 25 cause separation of the gas streams into a xenon-rich gas stream at one end 25 and the xenon-depleted carbon dioxide stream at the other end 26. The xenon-depleted gas stream passes through the one-way valve 20b to the pump 15 for recirculation. The volume of the system is controlled negative feedback from the pressure of the buffer vessel 21 acting on the carbon dioxide inlet valve 19.

The throttle at the xenon-rich outlet 25 of the vortex gas separator can be closed until there is sufficient xenon in the gas stream to allow separation and opened proportionally to the amount of xenon in the system. This concentration can be detected by ultrasound, katharometer or refractive index at any point from the selection valve 13a or 13b and the vortex tube 24.

The xenon-rich gas stream passes through a carbon dioxide absorber 27 and is stored in a vessel 28 ready for re-delivery to the patient circuit via a solenoid or piezo valve 4 under physician target electronic control and negative feedback from the patient gas detector 6 and a carbon dioxide absorber to remove any remaining carbon dioxide 29.

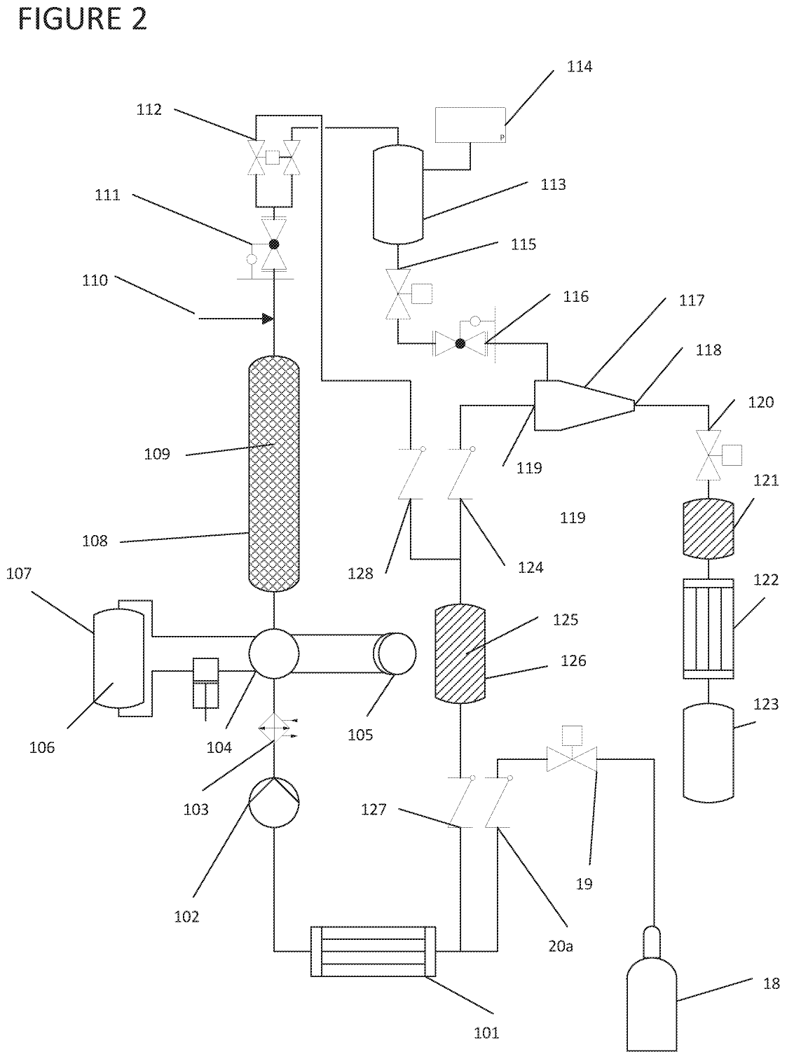

FIG. 2 shows the purification of xenon by liquid carbon dioxide.

Carbon dioxide contained in a pressurized cylinder with liquid and vapour phase 18 (approx. 55 bar at room temperature) passes through a powered valve 19 and one-way valve 20a to a condenser 101 to cool the carbon dioxide to −10 degrees Celsius, although other temperatures and pressures to ensure liquid carbon dioxide can be used. The cold liquid carbon dioxide passes to a liquid carbon dioxide pump 102 increasing the pressure to 70 bar although other liquid carbon dioxide pressures can be used. The fluid passes through a heater 103 to increase the temperature above the critical temperature of carbon dioxide, 31 degrees Celsius. In a preferred embodiment the fluid is heated to 50 degrees Celsius. The supercritical carbon dioxide passes to a rotary 6-port injection valve 104. This injection valve links to a fixed volume loop 105 that is filled with extracted xenon with contaminants from the patient or breathing system 106 contained in a pressurized vessel 107 at 70 bar and a temperature below 17 degrees Celsius such that the xenon is a liquid. Other temperatures and pressures to ensure liquid xenon can be used. The liquid xenon is pumped 108 around the loop during the filling setting of the rotary valve 104 and then during the load setting of the rotary valve 104, the valve turns and connects the loop to the flow of supercritical carbon dioxide from the pump 102. This flow takes the bolus of xenon/contaminants 106 into the chromatography column 108 filled with the stationary phase 109. In a preferred embodiment the stationary phase is plain silica although other normal and reverse-phase stationary phases can be used as knows to those skilled in the art.

The xenon 106 is separated from contaminants during passage through the column 108 by its interaction with the stationary phase 109, driven by the flow of carbon dioxide from the pump 102. The purified xenon, diluted in carbon dioxide, is detected by the detector 110 immediately after leaving the column. The detection method can be mass spectrometry, microthermal (katharometer), x-ray absorption, ultrasound or refractometry although other detection systems know to those familiar with the art can be used. When the bolus of xenon is detected, the electronic controller (not shown), often a Programmable Logic Controller, activates a three-way valve 112 to pass the xenon and carbon dioxide into the collection system. The xenon and carbon dioxide first pass through a back-pressure regulator 111 and then the three-way valve 112 into the collection buffer 113 with pressure sensor 114. When sufficient pressure is in the buffer 113, the xenon/CO₂ mixture passes through a powered valve 115 and pressure-reducing valve 116 into the vortex tube gas separator 117. The vortex tube gas separator separates the xenon from the carbon dioxide by the virtue of density, with the xenon exiting via the throttle valve at one end 118 and the carbon dioxide via the other end 119. The high xenon fraction coming from 118 is passed through soda lime 120 to remove any remaining carbon dioxide a powered valve 121 then a condenser 122 and stored in a vessel 123. This process may require increasing the pressure of the xenon (pump not shown). It is possible to use more than one vortex tube gas separator in series to increase the purity of the xenon fraction before soda lime.

The carbon dioxide leaves the vortex tube gas separator 119, passing through a one-way valve 124 and an activated carbon 125 filled capture chamber 126 to scrub out any contaminants and then passes through another one-way valve and back to the condenser 101 for recirculation.

Carbon dioxide exiting the column without xenon is passed through the back-pressure regulator 111, three-way valve 112 and is directed straight to recirculation via a one-way valve 128 and activated charcoal 125 filled capture chamber 126 to remove contaminants.

Further steps may be taken to remove microbiological contaminants, package and present the Xenon ready for re-supply as a medical gas. These steps are not shown but are familiar to those skilled in the art.

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention.

1-13. (canceled)

14. A method of reclaiming xenon anaesthetic agent, comprising:
i) passing gas including xenon anaesthetic agent from a medical environment through a filter so that xenon anaesthetic agent becomes bound to the filter;
ii) subjecting the filter to a supercritical fluid to carry the xenon anaesthetic agent from the filter; and
iii) separating the xenon anaesthetic agent from the supercritical fluid.

15. The method as claimed in claim 14, in which at step ii) the supercritical fluid is supercritical carbon dioxide and in which a mixture is formed in which both carbon dioxide and xenon are in a supercritical state.

16. A method as claimed in claim 14, further comprising the step of reintroducing the separated xenon anaesthetic agent to a patient.

17. An apparatus to recover xenon anaesthetic agent from a medical environment, comprising a container including a filter through which medical environment gas can be passed so that xenon anaesthetic agent can become reversibly bound thereto.

18. The apparatus as claimed in claim 17, in which the container is connected or connectable to a source of supercritical carbon dioxide for extraction of the xenon anaesthetic agent from the filter by supercritical carbon dioxide.

19. The apparatus as claimed in claim 18, further comprising a source of supercritical carbon dioxide.

20. The apparatus as claimed in claim 17, in which the container is connected or connectable to the exhaust port of an anaesthetic machine or medical device so that waste gas containing xenon is passed through the filter material in the container to bind the xenon gas from the waste gas stream.

21. The apparatus as claimed in claim 17, in which the container is tolerant of pressures in excess of the critical pressure of carbon dioxide.

22. The apparatus as claimed in claim 17, in which the container is intolerant to the critical pressure of carbon dioxide, the container can be placed in a pressure-tolerant container that is pressure-tolerant above the critical pressure of carbon dioxide.

23. The apparatus as claimed in claim 9, the apparatus further comprising a pressure-tolerant vessel that is pressure-tolerant above the critical pressure of carbon dioxide.

24. The apparatus as claimed in claim 17, in which the filter material comprises one or more of: aerogel, silica gel, zeolites, metal organic frameworks, metal doped silica/zeolite, metal doped aerogel.

25. The apparatus as claimed in claim 17, in which the container comprises a stainless-steel tube.

26. The apparatus as claimed in claim 17, comprising a tube with sealed, floating end caps to contain the filter material.

27. The apparatus as claimed in claim 17, further comprising means for separating xenon from carbon dioxide.

28. The apparatus as claimed in claim 17, further comprising a vortex tube.

29. The apparatus as claimed in claim 17, further comprising means for the chromatographic separation of xenon from contaminants.

30. The apparatus as claimed in claim 29, comprising one or more chromatography columns.

31. The apparatus as claimed in claim 17, further comprising soda lime for absorbing carbon dioxide.

32. The apparatus as claimed in claim 31, further comprising means for removing gaseous contaminants.

33. The apparatus as claimed in claim 17, further comprising means for removing microbiological contaminants.

34. The method of claim 14, comprising removing contaminants.
